(19) United States
(12) Patent Application Publication
(10) Pub. No.: US 2008/0241819 A1
(43) Pub. Date: Oct. 2, 2008

(54) METHOD AND APPARATUS FOR ENHANCED BACTERIOPHAGE-BASED DIAGNOSTIC ASSAYS BY SELECTIVE INHIBITION OF POTENTIAL CROSS-REACTIVE ORGANISMS

(75) Inventor: Breanna C. SMITH, Lyons, CO (US)

Correspondence Address: PATTON BOGGS LLP, 1801 CALFORNIA STREET, SUITE 4900, DENVER, CO, 80202, US

(73) Assignee: MicroPhage (TM) Incorporated, Longmont, CO (US)

(21) Appl. No.: 11/933,083

(22) Filed: Oct. 31, 2007

Related U.S. Application Data
(60) Provisional application No. 60/855,648, filed on Oct. 31, 2006.
(60) Provisional application No. 60/860,839, filed on Nov. 22, 2006.

(51) Int. Cl. C12Q 1/70

(52) U.S. Cl. 435/5

(57) ABSTRACT

A sample to be tested for the presence of a target microorganism is exposed to bacteriophage and conditions are provided to inhibit phage attachment to or replication in a potentially cross-reactive, non-target microorganism. The sample is incubated and assayed to detect the presence or absence of a bacteriophage marker to determine the presence or absence of the target microorganism. The inhibiting may comprise the addition of an inhibiting substance or the use of an inhibiting process. It may include inhibiting the growth of potentially cross-reactive bacteria while allowing growth of the target bacteria, selectively removing or blocking potential cross-reactive bacteria using selective binding agents or selectively destroying potentially cross-reactive bacteria.

### CROSS-REFERENCE TO RELATED APPLICATIONS

This Application is a Non-Provisional of Provisional (35 USC 119(e)) Application No. 60/855648 filed on Oct. 31, 2006. This Application is also a Non-Provisional of Provisional (35 USC 119(e)) Application No. 60/860839 filed on Nov. 22, 2006.

### FIELD OF THE INVENTION

The invention relates generally to the field of identification of microscopic living organisms, and more particularly to the identification of microorganisms using bacteriophage.

### BACKGROUND OF THE INVENTION

Bacteriophage are viruses that have evolved in nature to use bacteria as a means of replicating themselves. A bacteriophage (or phage) does this by attaching itself to a bacterium and injecting its genetic material into that bacterium, inducing phage replication. Some bacteriophage, termed lytic bacteriophage, rupture the host bacterium releasing the progeny phage into the environment to seek out other bacteria. The total incubation time for infection of a bacterium by parent phage, phage multiplication (amplification) in the bacterium to produce progeny phage, and release of the progeny phage after lysis can take as little as an hour depending on the phage, the host bacterium, and the environmental conditions of the sample.

Bacteriophage-based methods have been suggested as a method to accelerate microorganism identification. See, for example, U.S. Pat. No. 5,985,596 issued Nov. 16, 1999 and U.S. Pat. No. 6,461,833 B1 issued October 8, both to Stuart Mark Wilson; U.S. Pat. No. 4,861,709 issued Aug. 29, 1989 to Ulitzur et al.; U.S. Pat. No. 5,824,468 issued Oct. 20, 1998 to Scherer et al.; U.S. Pat. No. 5,656,424 issued Aug. 12, 1997 to Jurgensen et al.; U.S. Pat. No. 6,300,061 B1 issued Oct. 9, 2001 to Jacobs, Jr. et al.; U.S. Pat. No. 6,555,312 B1 issued Apr. 29, 2003 to Hiroshi Nakayama; U.S. Pat. No. 6,544,729 B2 issued Apr. 8, 2003 to Sayler et al.; U.S. Pat. No. 5,888,725 issued Mar. 30, 1999 to Michael F. Sanders; U.S. Pat. No. 6,436,652 B1 issued Aug. 20, 2002 to Cherwonogrodzky et al.; U.S. Pat. No. 6,436,661 B1 issued Aug. 20, 2002 to Adams et al.; U.S. Pat. No. 5,498,525 issued Mar. 12, 1996 to Rees et al.; Angelo J. Madonna, Sheila VanCuyk and Kent J. Voorhees, “Detection Of Esherichia Coli Using Immunomagnetic Separation And Bacteriophage Amplification Coupled With Matrix-Assisted Laser Desorption/Ionization Time-Of-Flight Mass Spectrometry”, Wiley InterScience, DOI:10.1002/rem.900, 24 Dec. 2002; and U.S. Patent Application Publication No. 2004/0224359 published Nov. 11, 2004. All of the foregoing references are hereby incorporated by reference to the same extent as though fully disclosed herein.

In each of these methods, samples potentially containing target bacteria are incubated with bacteriophage specific for those bacteria. In the presence of the bacteria, the bacteriophage infect and replicate in the bacteria resulting in the production of a measurable signal, above input phage levels, indicating the presence of the target bacteria. Some methods utilize the detection of progeny phage released from infected target bacteria as a means of detection and identification. In this case, progeny phage are not produced if the parent phage do not successfully infect the target bacteria. Still other methods rely on the detection of phage replication products rather than whole progeny phage. For example, luciferase reporter bacteriophage produce luciferase when they successfully infect target bacteria. The luciferase then produces light that, if detected, indicates the presence of target bacteria in the sample. Other methods rely on the detection of bacterial debris that is released following a successful lytic infection of target bacteria by a specific bacteriophage. To accurately identify the target bacteria, each of these phage-based diagnostic methods demand that the bacteriophage have both high sensitivity for the target bacteria and high specificity to avoid replication in non-target strains or species of bacteria. Finding or developing bacteriophage with those characteristics can be very challenging. Bacteriophage with high level sensitivity often lack sufficient specificity, i.e., they cross react with non-target bacteria. Thus, there remains a need for microorganism detection methods using bacteriophage that achieves higher levels of specificity while retaining high-level sensitivity.

### BRIEF SUMMARY OF THE INVENTION

The invention solves the above problems, as well as other problems of the prior art, by identifying conditions wherein phage attachment or replication in potentially cross-reactive, non-target bacteria is inhibited in some manner while minimally affecting attachment or replication in the target bacteria. This inhibition can be accomplished in at least three ways: 1) inhibiting the growth of potentially cross-reactive bacteria while allowing growth of the target bacteria; 2) selectively removing or blocking potential cross-reactive bacteria using selective binding agents; and 3) selectively destroying potentially cross-reactive bacteria. Other methods with the same results can be contemplated by those skilled in the arts.

The invention provides a method of determining the presence or absence of a target microorganism in a sample to be tested, the method comprising: (a) combining with the sample an amount of bacteriophage capable of attaching to the target microorganism to create a bacteriophage-exposed sample; (b)providing conditions to the bacteriophage-exposed sample sufficient to allow the bacteriophage to attach to the target microorganism while inhibiting phage attachment or replication in a potentially cross-reactive, non-target microorganism; and (c) assaying the bacteriophage-exposed sample to detect the presence or absence of a bacteriophage marker to determine the presence or absence of the target microorganism. Preferably, the method comprises conditions to permit the bacteriophage to infect the target microorganism and to multiply in the target microorganism while eliminating or inhibiting phage replication in potentially cross-reactive microorgansims. Preferably, the method further comprises a bacteriophage marker with a detectable tag, and wherein the assaying comprises performing a target separation process, the separation process capable of separating the bacteriophage-exposed sample into a target microorganism portion containing target microorganisms present in the sample and an unbound tagged bacteriophage portion containing tagged bacteriophage that are not bound to the target microorganism. Preferably, the inhibiting comprises inhibiting the growth of a potentially cross-reactive bacterium while allowing growth of a target bacterium. Preferably, the inhibiting comprises adding an inhibiting substance to the sample. Preferably, the inhibiting substance is selected from the group consisting of divalent cations, antibiotics, chelators, and metal compounds. Preferably, the inhibiting comprises selectively removing a potential cross-reactive microorganism from the sample using a selective binding agent attached to a substrate. Preferably, the selective removal comprises using an antibody or bacteriophage selective for the non-target bacteria. Preferably, the substrate comprises microparticles. Preferably, the inhibiting comprises selectively destroying or significantly slowing the growth of a potentially cross-reactive microorganism. Preferably, the destroying comprises selective combining of antibiotics with the sample. Preferably, the destroying comprises combining with the sample bacteriophage that selectively bind to and/or infect one or more potentially cross-reactive, non-target microorganisms. Preferably, the assaying comprises an immunological assay.

The invention also provides a selective growth medium for determining the presence or absence of a target microorganism in a sample to be tested, the medium comprising a combination of one or more bacteriophage specific to the target microorganism, a nutritional growth medium, and an inhibiting substance(s) that inhibits phage attachment to or replication in a potentially cross-reactive, non-target microorganism. Preferably, the inhibiting substance(s) is selected from the group consisting of bacteriophage specific to the cross-reactive microorganism, antibodies, antibiotics, antibacterial compounds, divalent cations, chelators, and metal compounds.

In addition, the invention provides a kit for determining the presence or absence of a target microorganism in a sample to be tested, the kit comprising a selective growth medium containing: one or more bacteriophage specific to the target microorganism, a nutritional growth medium, and an inhibiting substance. Preferably, the bacteriophage and inhibiting substance can be combined in a single container. Alternatively, the bacteriophage and the nutritional growth medium may be in one container and the inhibiting substance in another. Preferably, the kit further includes a rapid diagnostic tool, such as a lateral flow strip.

In addition, the invention provides a kit for determining the presence or absence of a target microorganism in a sample to be tested, the kit comprising: a bacteriophage capable of attaching to or infecting the target microorganism, a nutritional growth medium; and an inhibitor substance capable of inhibiting phage attachment or replication in a potentially cross-reactive, non-target microorganism. Preferably, the bacteriophage and growth medium is in a first container and the inhibitor substance is in a second container. Preferably, the kit further includes a rapid diagnostic tool, such as a lateral flow strip.

The invention solves the problem of increasing the specificity of phage-based microorganism detection methods without appreciably altering the sensitivity to target microorganisms. Numerous other features, objects, and advantages of the invention will become apparent from the following detailed description when read in conjunction with the accompanying drawings.

### BRIEF DESCRIPTION OF THE DRAWINGS

FIG. 1 illustrates a phage amplification process;

FIG. 2 illustrates several exemplary embodiment of a process and apparatus for determining the presence or absence of a microorganism according to the invention; and

FIG. 3 illustrates a detection kit according to the invention.

### DETAILED DESCRIPTION OF THE INVENTION

The invention provides methods and apparatus to enhance the detection of microorganisms using bacteriophage. As known in the art, bacteriophage generally are specific to a particular microorganism; however, they often demonstrate some low-level cross reactivity against organisms that are closely related. Generally, bacteriophage are used to detect the presence of a target microorganism in a sample by combining bacteriophage specific to the target microorganism. The bacteriophage-exposed sample then is incubated by providing conditions sufficient to allow bacteriophage to attach to or replicate in said target microorganism. After incubation, the bacteriophage-exposed sample is assayed to detect the presence or absence of a bacteriophage marker to determine the presence or absence of said target microorganism. Successful phage attachment or replication in a sample indicates the presence of the target microorganism in the sample. The present invention addresses the fact that bacteriophage may not be completely specific to a particular microorganism. The related non-target microorganisms with which a bacteriophage will interact are generally called “cross-reactive” organisms.

Methods have been developed in the art to determine if a bacteriophage has attached to or has replicated within a microorganism. Generally, these involve detection of a flag attached to the bacteriophage, detection of the bacteriophage itself, detection of a portion of the bacteriophage such as a major capsid protein, detection of an enzyme or other byproduct of phage replication particularly with modified reporter bacteriophage such as a luciferase reporter phage, or detection of bacterial debris released after successful phage replication and microorganism lysis. For the purposes of this invention, detection of any of these phage related signals comprises detection of a phage marker.

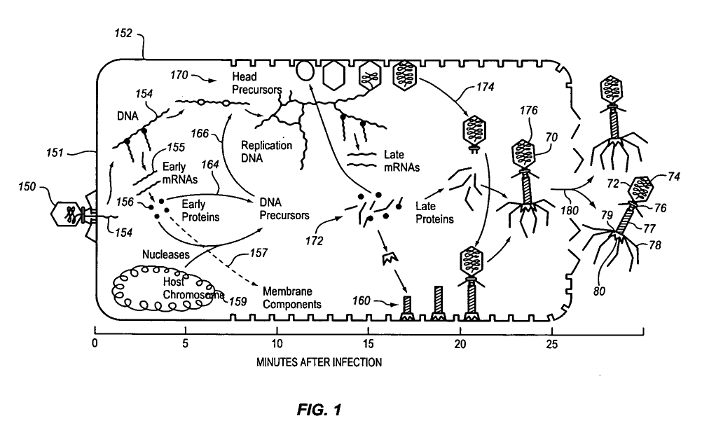

The invention can be more readily grasped with an understanding of some of the details of bacteriophage replication. The replication process is illustrated in FIG. 1. A typical bacteriophage 70, in this case MS2-E. Coli, is shown emerging from a bacterium 152 to the right of the figure. Structurally, a bacteriophage 70 comprises a protein shell or capsid 72, sometimes referred to as a head, that encapsulates the viral nucleic acids 74, i.e., the DNA and/or RNA. A bacteriophage may also include proteins making up the capsid or the DNA/RNA, a neck 76, a tail sheath 77, tail fibers 78, an end plate 79, and pins 80. The capsid 72 is constructed from repeating copies of one or more proteins. Referring to the left of the figure, when a phage 150 infects a bacterium 152, which in this case is a cross-reactive bacterium, it attaches itself to a particular site on the bacterial wall or membrane 151 and injects its nucleic acid 154 into that bacterium, inducing it to replicate the phage from tens to thousands of copies. The process is shown in schematic in FIG. 1. The DNA evolves to early mRNAS 155 and early proteins 156, some of which become membrane components along line 157 and others of which utilize bacteria nucleases from host chromosomes 159 to become DNA precursors along line 164. Others migrate along the direction 170 to become head precursors that incorporate the DNA along line 166. The membrane components evolve along the path 160 to form the sheath, end plate, and pins. Other proteins evolve along path 172 to form the tail fibers. When formed, the head releases from the membrane 151 and joins the tail sheath along path 174, and then the tail sheath and head join the tail fibers at 176 to form the bacteriophage 70. Some bacteriophage, termed lytic bacteriophage, rupture the host bacterium, shown at 180, releasing the progeny phage into the environment to seek out other bacteria in environment. Any process or substance that will selectively interfere with the attachment or replication process in a cross-reactive bacterium 152 is contemplated as forming part of the invention. This includes any process or substance that directly inhibits phage attachment or replication as well as any process or substance that affects the bacterium 152 itself, and thereby indirectly inhibits phage attachment or replication.

The invention identifies conditions wherein phage attachment and/or replication in potentially cross-reactive, non-target bacteria is inhibited in some manner, and uses this inhibition to increase the specificity of the phage-based diagnostic process. The inhibiting may comprise the addition of an inhibiting substance or the use of an inhibiting process. Three embodiments of the inhibition process are described herein: 1) inhibiting the growth of potentially cross-reactive bacteria while allowing growth of the target bacteria; 2) selectively removing or blocking potential cross-reactive bacteria using selective binding agents; and 3) selectively destroying potentially cross-reactive bacteria. These embodiments are intended to be illustrative, though the invention is not limited to these embodiments. Other methods with the same results can be contemplated by those skilled in the arts.

Inhibition of potentially cross-reactive bacteria can be accomplished using methods common to microbiological detection. For example, salts such as sodium chloride (in high concentration), divalent cations, antibiotics such as Polymyxin B or E, antiseptics such as acriflavine, metal compounds such as potassium tellurite, and iron chelators such as desferoxamine inhibit the growth of some coagulase negative Staphylococcus (CNS) while allowing the growth of Staphylococcus aureus. These compounds can also significantly inhibit or retard replication of bacteriophage in CNS while minimally affecting replication in Staphylococcus aureus. The usage of selective media to differentially affect the efficiency and timing of phage replication is a novel method for improving the specificity of bacteriophage-based bacterial diagnostic methods.

Removal or blocking of non-target bacteria may be accomplished using antibodies, bacteriophage selective for the non-target bacteria, or other compounds that selectively bind to non-target bacteria. For a Staphylococcus aureus identification test, removal of CNS species can be beneficial. Binding of these compounds to non-target bacteria may be sufficient to block subsequent binding to those bacteria by bacteriophage that are inadequately specific for the target bacteria, thus preventing non-specific infection and replication in non-target bacteria. Alternatively, these compounds may be attached to other substrate such as microparticles, magnetic beads, or solid substrates. When incubated with a sample, potential non-target bacteria will selectively bind to the substrate. The substrate then can be physically removed from the sample. Separation methods include centrifugation of microparticles, application of a magnetic field for isolating magnetic beads, or other separation process.

Selective destruction of non-target bacteria can be accomplished using antibacterial compounds that selectively destroy non-target bacteria such that they are not susceptible to phage infection while leaving target bacteria largely unharmed and susceptible to phage infection. Such compounds include a) selective antibiotics and b) bacteriophage that selectively bind to and/or infect potentially cross-reactive, non-target bacteria. The latter are complimentary bacteriophage to the primary bacteriophage used to selectively infect the target bacteria in the sample. Complimentary bacteriophage can destroy non-target bacteria by successfully infecting and lysing those non-target bacteria such that phage infection by the primary bacteriophage is eliminated or significantly reduced. Complimentary bacteriophage can also be used to destroy non-target bacteria by a process known as lysis from without. Lysis from without refers to the destruction of a bacterium when hundreds or thousands of phage particles bind to its cell wall. This process can be utilized in this invention by adding a high concentration of complimentary phage to the sample such that large numbers of complimentary phage quickly and selectively bind to potentially cross-reactive bacteria. Under pressure of multiple phage binding, the cross-reactive bacteria can be made to burst, eliminating them as a focus for phage infection by the prime bacteriophage.

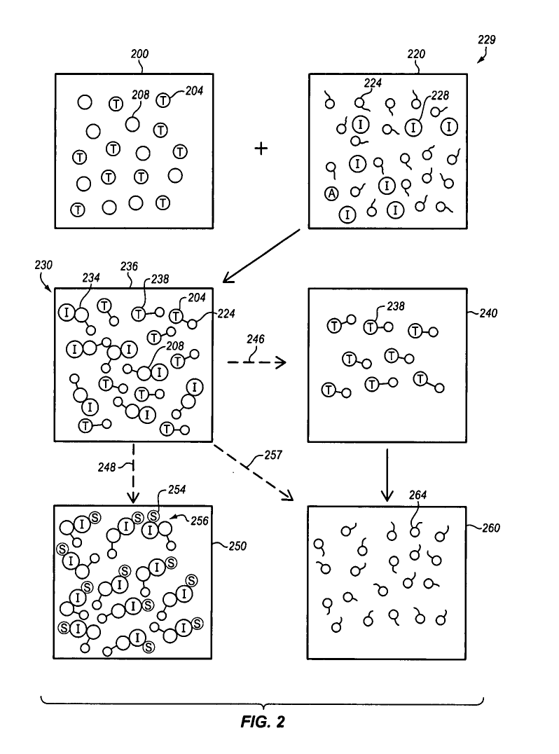

FIG. 2 illustrates exemplary processes according to the invention as well as exemplary bacteriophage-inhibitor combinations. Sample 200 contains both a target bacteria 204 and a cross-reactive bacteria 208. The additive combination 220 comprises a bacteriophage 224 and a inhibitor component 228, identified by an “I”. Preferably, the components 224 and 228 are provided in a medium suitable for the bacteriophage 224 and inhibitor 228, such as a liquid or some other culture medium. Bacteriophage 224 is specific to target bacteria 204, but also can attach to and may also infect cross-reactive bacteria 208. Inhibitor 228 may be another bacteriophage, an antibody, a chemical, an antibiotic, an antibacterial compound, or any other substance that can act to inhibit the cross-reactive contribution to the assay for the bacteriophage marker.

Additive Combination 220 is added to sample 200 as shown at 229. In the exposed sample 236, the bacteriophage 224 find and attach to both the target bacteria 204 and the cross-reactive bacteria 208. For simplicity, only one bacteriophage is shown attached to each bacterium although many, often in the hundreds, will attach. In addition the inhibitor component 228 will also affect the cross-reactive bacteria, either by attaching to it, if the inhibitor is a bacteriophage or antibody, or by some other interaction as indicated at 234. Whatever the interaction is, it will negatively affect the cross-reactive bacteria. For example, if the inhibitor component 228 is a bacteriophage that is specific to the cross-reactive bacteria but not to the target bacteria, then there could well be a thousand or more bacteriophage attaching to the cross-reactive bacteria, which will destroy the bacteria such that the bacteriophage 224 do not replicate and do not create bacteriophage markers that contribute to the assay; or, if the inhibitor is an antibody or a bacteriophage, the antibody or bacteriophage can be used to attach the cross-reactive bacteria to a substrate 254 to create a substrate/inhibitor/cross-reactive bacteria complex 256 which can be removed from the sample via path 148 to isolate the cross-reactive bacteria at 250. In this case, the target bacteria process will proceed via path 246 to 240, in which essentially only the target bacteria/bacteriophage complexes 238 will remain in the sample, and the bacteriophage will create a bacteriophage marker, such as 264, in the sample 260 which will greatly enhance the specificity of the assay because the cross-reactive bacterial will not contribute to the marker assay; or, if the inhibitor is an antibiotic or chemical that is specific to the cross-reactive bacteria, then the cross-reactive bacteria will die, and the bacteriophage 224 attached to the cross-reactive bacteria will not be able to replicate. Thus, the result is the same, though via a slightly different path 257: the sample 260 contains essentially only markers 264 from the target bacteria and only these will contribute to the marker assay. In 260, the marker is shown as a progeny bacteriophage, but there can also be many other types of phage markers. The detection assay may use immunoassay methods utilizing antibody-binding events to produce detectable signals such as ELISA, radioimmunoassay, lateral flow immunochromatography (LFI), and flow-through assay technology. The detection assay may also use flow cytometry, western blots, aptamer-based assays, immunofluoresence, matrix-assisted laser desorption/ionization with time-of-flight mass spectrometry (MALDI-TOF-MS), referred to herein as MALDI, and other detection methods.

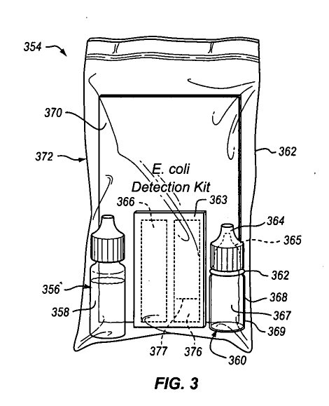

FIG. 3 shows an exemplary test kit 354 for detecting a microscopic living organism. Test kit 354 preferably includes a container 356 of cross-reactive microorganism inhibitor solution 358, a reaction container 360, one or more detection elements 366 enclosed in a protective case 363, directions 370 for using the kit, and a receptacle 372 for holding the foregoing test kit parts. Protective case 363 may also include a reference detection element 376 indicating the expected result 367 if no bacteria are present. For example, if the detection element is an immunoassay test device such as a lateral flow strip or a flow-through device, the reference detection element may be an identical immunoassay device on which a reference sample of bacteriophage has been applied which had no bacteria present. Reaction container 360 includes a container body 367 and a container closure 364. Preferably, the reaction container body is a bottle 367 and the reaction container closure is a bottle cap 364. Reaction container 360 contains phage 368 and an optional growth medium 369. Phage 368 preferably comprises a predetermined amount of phage that is attached to the interior wall 369 of reaction container body 367. Cap 364 is preferably a screw-on cap having interior threads 362 that mate with threads on the top portion of bottle 367. Cap 364 preferably includes a dispenser 365, which preferably is a dropper head designed to release drops of a predetermined size. In this embodiment, detection element 366 comprises an antibody, and more specifically is a lateral flow strip 366, but it also could be a flow-through device or any other detection apparatus. Preferably, receptacle 372 comprises a plastic bag 372, which serves the dual purpose of holding the test kit parts and providing a convenient disposal receptacle after the test is completed. Optionally, test kit 354 may be simplified by adding cross-reactive microorganism inhibitor solution 358 to reaction container 360 thereby eliminating container 356.

The methods of the invention were developed to allow bacteriophage with high sensitivity but less than optimal specificity to be used in phage-based methods for identifying bacteria. It allows for the differentiation of closely related bacterial strains from the target strain. It minimizes the need for high specificity that is difficult to achieve with bacterial species that are closely related to the target species.

The use of selective compounds to inhibit certain species of bacteria have been published in the scientific literature since the 1950s; however, none of this literature contemplates the use inhibition to hinder the replication of bacteriophage attachment or replication. Those skilled in the art will recognize that the present disclosure enables the use of any of the inhibition methods of the prior art used in inhibition of bacteria can potentially be used to inhibit bacteriophage replication.

Many other phage-based methods and apparatus used to identify the microorganism and/or to determine the antibiotic resistance test or antibiotic susceptibility can be enhanced by the method and apparatus of the invention. For example, a phage amplification process, such as a process described in U.S. Patent Application Publication No. 2005/0003346 entitled “Apparatus And Method For Detecting Microscopic Living Organisms Using Bacteriophage” may be enhanced by the present invention; or a process of attaching to a microorganism, such as described in PCT Patent Application No. PCT/US06/12371 entitled “Apparatus And Method For Detecting Microorganisms Using Flagged Bacteriophage” may also be enhanced. The foregoing patent applications are hereby incorporated herein by reference to the same extent as though fully disclosed herein.

Any other phage-based identification process may also be used. Examples of such processes are disclosed in the following publications:

United States patents:

U.S. Pat. No. 4,104,126 issued Aug. 1, 1978 to David M. Young

U.S. Pat. No. 4,797,363 issued Jan. 10, 1989 to Teodorescu et al.

U.S. Pat. No. 4,861,709 issued Aug. 29, 1989 to Ulitzur et al.

U.S. Pat. No. 5,085,982 issued Feb. 4, 1992 to Douglas H. Keith

U.S. Pat. No. 5,168,037 issued Dec. 1, 1992 to Entis et al.

U.S. Pat. No. 5,498,525 issued Mar. 12, 1996 to Rees et al.

U.S. Pat. No. 5,656,424 issued Aug. 12, 1997 to Jurgensen et al.

U.S. Pat. No. 5,679,510 issued Oct. 21, 1997 to Ray et al.

U.S. Pat. No. 5,723,330 issued Mar. 3, 1998 to Rees et al.

U.S. Pat. No. 5,824,468 issued Oct. 20, 1998 to Scherer et al.

U.S. Pat. No. 5,888,725 issued Mar. 30, 1999 to Michael F. Sanders

U.S. Pat. No. 5,914,240 issued Jun. 22, 1999 to Michael F. Sanders

U.S. Pat. No. 5,958,675 issued Sep. 28, 1999 to Wicks et al.

U.S. Pat. No. 5,985,596 issued Nov. 16, 1999 to Stuart Mark Wilson

U.S. Pat. No. 6,090,541 issued Jul. 18, 2000 to Wicks et al.

U.S. Pat. No. 6,265,169 B1 issued Jul. 24, 2001 to Cortese et al.

U.S. Pat. No. 6,300,061 B1 issued Oct. 9, 2001 to Jacobs, Jr. et al.

U.S. Pat. No. 6,355,445 B2 issued Mar. 12, 2002 to Cherwonogrodzky et al.

U.S. Pat. No. 6,428,976 B1 issued Aug. 6, 2002 to Chang et al.

U.S. Pat. No. 6,436,652 B1 issued Aug. 20, 2002 to Cherwonogrodzky et al.

U.S. Pat. No. 6,436,661 B1 issued Aug. 20, 2002 to Adams et al.

U.S. Pat. No. 6,461,833 B1 issued Oct. 8, 2002 to Stuart Mark Wilson

U.S. Pat. No. 6,524,809 B1 issued Feb. 25, 2003 to Stuart Mark Wilson

U.S. Pat. No. 6,544,729 B2 issued Apr. 8, 2003 to Sayler et al.

U.S. Pat. No. 6,555,312 B1 issued Apr. 29, 2003 to Hiroshi Nakayama

### United States Published Applications:

2002/0127547 A1 published Sep. 12, 2002 by Stefan Miller

2004/0121403 A1 published Jun. 24, 2004 by Stefan Miller

2004/0137430 A1 published Jul. 15, 2004 by Anderson et al.

2005/0003346 A1 published Jan. 6, 2005 by Voorhees et al.

### Foreign Patent Publications:

EPO 0 439 354 A3 published Jul. 31, 1991 by Bittner et al.

WO 94/06931 published Mar. 31, 1994 by Michael Frederick Sanders

EPO 1 300 082 A2 published Apr. 9, 2003 by Michael John Gasson

WO 03/087772 A2 published Oct. 23, 2003 by Madonna et al.

### Other Publications:

Favrin et al., “Development and Optimization of a Novel Immunomagnetic Separation-Bacteriophage Assay for Detection of Salmonella enterica Serovar Enteritidis in Broth”, Applied and Environmental Microbiology, January 2001, pp. 217-224, Volume 67, No. 1.

All of the forgoing publications are hereby incorporated by reference to the same extent as though fully disclosed herein. Any other bacteriophage-based process may be used as well.

There has been described a microorganism detection method which is specific to a selected organism, sensitive, simple, fast, and/or economical, and having numerous novel features. It should be understood that the particular embodiments described within this specification are for purposes of example and should not be construed to limit the invention, which will be described in the claims below. Further, it is evident that those skilled in the art may now make numerous uses and modifications of the specific embodiment described, without departing from the inventive concepts. Equivalent structures and processes may be substituted for the various structures and processes described; the subprocesses of the inventive method may, in some instances, be performed in a different order; or a variety of different materials and elements may be used. Consequently, the invention is to be construed as embracing each and every novel feature and novel combination of features present in and/or possessed by the microorganism detection apparatus and methods described.

What is claimed as new and desired to be protected by Letters Patent of the United States is:

1. A method of determining the presence or absence of a target microorganism in a sample to be tested, said method comprising:
(a) combining with said sample an amount of bacteriophage capable of attaching to said target microorganism to create a bacteriophage-exposed sample;
b) providing conditions to said bacteriophage-exposed sample sufficient to allow said bacteriophage to attach to said target microorganism while inhibiting phage attachment or replication in a potentially cross-reactive, non-target microorganism; and
(c) assaying said bacteriophage-exposed sample to detect the presence or absence of a bacteriophage marker to determine the presence or absence of said target microorganism.

2. A method as in claim 1 wherein said microorganism is a bacterium and said assaying comprises detecting said bacteriophage marker as an indication of the presence of said target bacterium in said sample.

3. A method as in claim 1 wherein said providing comprises providing conditions to permit said bacteriophage to infect said target microorganism and to replicate in said target microorganism.

4. A method as in claim 1 wherein said inhibiting comprises inhibiting the growth of a potentially cross-reactive bacterium while allowing growth of a target bacterium.

5. A method as in claim 4 wherein said inhibiting comprises adding an inhibiting substance to said sample.

6. A method as in claim 5 wherein said inhibiting substance is selected from the group consisting of sodium chloride, divalent cations, iron chelators, antibiotics, and metal compounds.

7. A method as in claim 1 wherein said inhibiting comprises selectively removing a potential cross-reactive microorganism from said sample using a selective binding agent attached to a substrate.

8. A method as in claim 7 wherein said selective removal comprises using an antibody or bacteriophage selective for the non-target bacteria.

9. A method as in claim 7 wherein said substrate comprises microparticles.

10. A method as in claim 1 wherein said inhibiting comprises selectively destroying a potentially cross-reactive microorganism.

11. A method as in claim 10 wherein said destroying comprises combining selective antibiotics with said sample.

12. A method as in claim 10 wherein said destroying comprises combining with said sample bacteriophage that selectively bind to and/or infect one or more potentially cross-reactive, non-target microorganisms.

13. A method as in claim 1 wherein said assaying comprises an immunological assay.

14. A medium for determining the presence or absence of a target microorganism in a sample to be tested, said medium comprising a bacteriophage specific to said target microorganism and an inhibiting substance that inhibits attachment of the bacteriophage to or replication in a potentially cross-reactive, non-target microorganism.

15. A medium as in claim 14 wherein said inhibiting substance is selected from the group consisting of bacteriophage specific to said cross-reactive microorganism, an antibody, an antibiotic, an antibacterial compound, and a chemical.

16. A medium as in claim 15 wherein said chemical is selected from the group consisting of sodium chloride and metal compounds.

17. A kit for determining the presence or absence of a target microorganism in a sample to be tested, said kit comprising: a bacteriophage capable of attaching to or infecting said target microorganism; and an inhibitor substance capable of inhibiting phage attachment or replication in a potentially cross-reactive, non-target microorganism.

18. A kit as in claim 17 wherein said bacteriophage is in a first container and said inhibitor substance is in a second container.

19. A kit as in claim 17 wherein said bacteriophage and inhibitor substance are in a single container.

20. A kit as in claim 17 and further including an immunoassay test device.
